(19) United States
(12) Patent Application Publication
(10) Pub. No.: US 2002/0168288 A1
(43) Pub. Date: Nov. 14, 2002

(54) Heating sludge

(76) Inventor: Colin Edward Brade, Nottinghamshire (GB)

Correspondence Address: SMITH-HILL AND BEDELL, 12670 N W BARNES ROAD, SUITE 104, PORTLAND, OR, 97229

(21) Appl. No.: 10/142,551

(22) Filed: May 8, 2002

Related U.S. Application Data
(63) Continuation-in-part of application No. 09/844,023, filed on Apr. 27, 2001, now abandoned.

(30) Foreign Application Priority Data
May 8, 2001 (GB) 0111167.3

(51) Int. Cl. A61L 2/08

(52) U.S. Cl. 422/26

(57) ABSTRACT

A method and apparatus for treating sewage sludge by heating in a sludge pasteurisation process in which small bubbles of steam are injected into a continuous flow of sludge to cause highly turbulent sludge flow conditions during which the steam condenses to heat the sludge. Incoming, untreated sludge is initially pre-heated as required and as available heat sources such as independent power plant or excess capacity of the steam generating boiler. Thus, a suitable treating temperature is more readily achieved. The heated sludge is retained in an insulated tank to allow the pasteurisation process to be completed. Heat dumping may be provided to regulate treatment temperatures.

The present invention concerns heating sludge, especially but not exclusively the heat treatment of sewage sludge.

The main aim of the heat treatment of sludge is to pasteurise the sludge.

Traditionally, hot fluids have been used for heat treatment of sludge. Steam has the benefit that high temperatures can be achieved by direct application without separation through a heat exchanger surface. Hot water or other fluids require a heat exchanger and at high temperatures sludge tends to bake onto the sludge side of the heat exchanger surface. Thus, in practice, there is a limit to hot water temperature of about 80° C. and this in turn requires a large heat exchanger to achieve at least a 70° C. sludge treatment temperature. Very large heat exchangers by their nature use a considerable amount of energy for pumping, etc. Unfortunately, steam has its own disadvantages in that an appropriate steam source may not be readily available at the treatment site and addition of steam to a sludge flow may increase the processed sludge volume by 8% with added transport, storage and disposal problems.

It will be appreciated that there may be a number of heat sources available as a treatment site which are not being utilised fully. In such circumstances, in order to improve thermal efficiency and conservation there is a desire to maximise heat usage from all heat sources conveniently available. Such surplus heating capacity will generally not be in the form of steam. In such circumstances, there is an objective to use steam judiciously and to its maximum effect.

According to the present invention there is provided a method of treating sludge by continuously passing a flow of sludge through a thermal reactor with a regulated injection of steam into the flow of sludge in order to achieve a treatment temperature, the rate of sludge flow and manner of steam injection being such that treatment conditions are provided in the vessel, removing heated sludge from the vessel and replacing the removed sludge by untreated sludge, incoming untreated sludge being pre-heated as required from any available heat source through heat exchange with an appropriate transfer medium in order to regulate, and normally limit, the volume of steam injection required to achieve the treatment temperature in the flow of sludge or the speed of attainment of the treatment temperature.

Preferably the sludge is caused to flow in a continuous pipework loop from a sludge inlet to a sludge outlet spaced from the inlet, steam being injected into the sludge downstream of the sludge inlet and closer to the inlet than the outlet.

Preferably the volume of steam is of the order of one hundred times that of the sludge into which it is injected.

Preferably the steam is injected into the sludge in the form of small bubbles, that is bubbles of around 200 micron diameter.

Preferably the steam is injected at a pressure of about 2 bar absolute and a quantity of around 1000 kilograms per hour, the sludge input/output being around 3 liters per second, the pipe diameter around 100 mm and the rate of flow through the pipe around 20 liters per second.

Preferably treated sludge is collected sequentially in at least three collection tanks. Preferably while one tank is filling another is emptying and a third is storing heated sludge during its heat treatment cycle.

Preferably the tanks are maintained at atmospheric pressure.

Preferably sludge is fed to the tanks at or near the bottom of the tanks. Preferably the air discharging from a filling tank is fed to an emptying tank.

Preferably treated sludge discharged from a tank is cooled in a two stage process in separate heat exchangers.

Preferably heat is removed from treated sludge in a heat exchanger and transfers to unprocessed untreated sludge to be fed to the treatment vessel.

The step of pre-heating the sludge may include heating the sludge with hot water, which may be from a boiler which may also be the steam boiler with suitable steam to water condensing equipment, an engine, a combined heat and power plant or any other suitable heating means.

Hot treated sludge may be subject to a heat transfer step to recover heat to the incoming sludge, preferably prior to the pre-heating step. The heat recovery step may comprise arranging the heated sludge in a heat transfer relation with a heat absorption medium, for example water. The step may further include arranging untreated incoming sludge in heat transfer relation with said heat absorption medium. Heat may be transferred from the treated sludge to the heat absorption medium and thereafter to the untreated incoming sludge. The temperature of the cooled treated sludge is regulated during the heat recovery step by removing heat from the heat absorption media to a heat dump medium which may be water or air.

Further according to the present invention there is provided apparatus for treating sludge comprising a thermal reactor, means for circulating sludge continuously around the reactor as a flow of sludge, means for regulated injection of steam into the flow of sludge, means for removing treated sludge from reactor and means for replacing removed heated sludge by untreated sludge, the apparatus also includes a heat exchange assembly whereby heat is transferred between the treated sludge and the incoming untreated sludge within pre-heating means which heats the incoming untreated sludge before that sludge is heated in the thermal reactor to regulate the volume of steam required to be injected into the flow of sludge in order to heat that sludge in the reactor to a treatment temperature or the speed of attainment of that treatment temperature by the sludge.

Preferably the treatment vessel is a continuous pipework loop having an inlet and at least one outlet spaced from the inlet, a steam injector for providing heating steam to the sludge flow in the form of small bubbles being provided adjacent the inlet.

Preferably three collection tanks are provided for collecting heated sludge, each tank having inlet and outlet means and control means for said inlet and outlet means whereby as one tank is being filled with heated sludge, another is being emptied of heat treated sludge, the or each remaining tank storing the sludge for complete heat treatment during said filling/emptying process.

Preferably said tanks are maintained at atmospheric pressure by providing a connection between atmosphere and the air space at the top of each tank.

Preferably the sludge inlets and outlets are located at or near the bottom of each tank.

Preferably heat exchange means are provided in the outlet line from said tanks.

Possibly, the two heat exchangers comprise a combined heat exchange assembly, whereby heat is transferred between the treated sludge and the untreated sludge and a cooling medium.

Preferably, the heat exchange assembly comprises a first heat exchanger in which sludge can be arranged in thermal communication with a heat transfer medium. A first line may be provided to feed treated sludge to the first heat exchanger to transfer heat from the treated sludge to the heat transfer medium. The heat exchange assembly may further include a second heat exchanger in which sludge can be arranged in thermal communication with the heat transfer medium. A second line may be provided to feed untreated sludge to the second heat exchanger to transfer heat from the heat transfer medium to the untreated sludge.

Preferably, the heat transfer assembly includes circulating means to circulate the heat transfer medium between the first and second heat exchangers. The heat transfer medium may be a fluid, for example water.

The heat transfer assembly may further include heat dump means in which the aforesaid heat transfer medium can be arranged in thermal communication with a heat dump medium, in which heat can be transferred from the heat transfer medium to the heat dump medium. The heat dump means may include a heat exchange plate across which heat transfer from the aforesaid heat transfer medium can occur. The heat dump medium maybe a heat transfer fluid, for example water or air.

The circulating means may be arranged to circulate the heat transfer medium through the first heat exchanger, the second heat exchanger and the heat dump means.

The apparatus may include pre-heating means for heating the untreated sludge before the sludge is heated in the thermal reactor. The pre-heating means is preferably provided upstream of the thermal reactor. The pre-heating means may include means for heating a heating medium, and may further include a heat exchange means for transferring heat from the heating medium to the untreated sludge. Steam means may also be provided to feed steam to the heated heating medium. The steam feed means may be arranged in fluid communication with said steam injection means.

The apparatus may include heat transfer means to transfer heat from the treated sludge to the untreated sludge. The heat transfer means may include the aforesaid second heat exchanger.

Preferably the pipework loop is arranged alongside the tanks at or near the base thereof.

An embodiment of the present invention will now be described by way of example only with reference to the accompanying drawings in which:

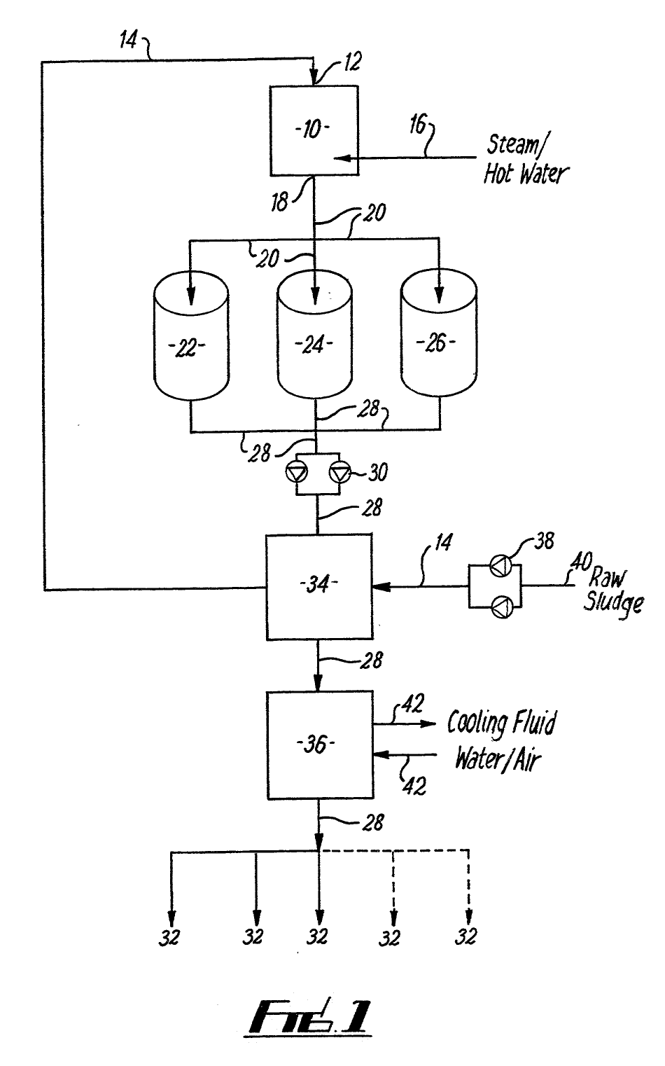

FIG. 1 shows a block diagram of a previous sludge heat treatment apparatus;

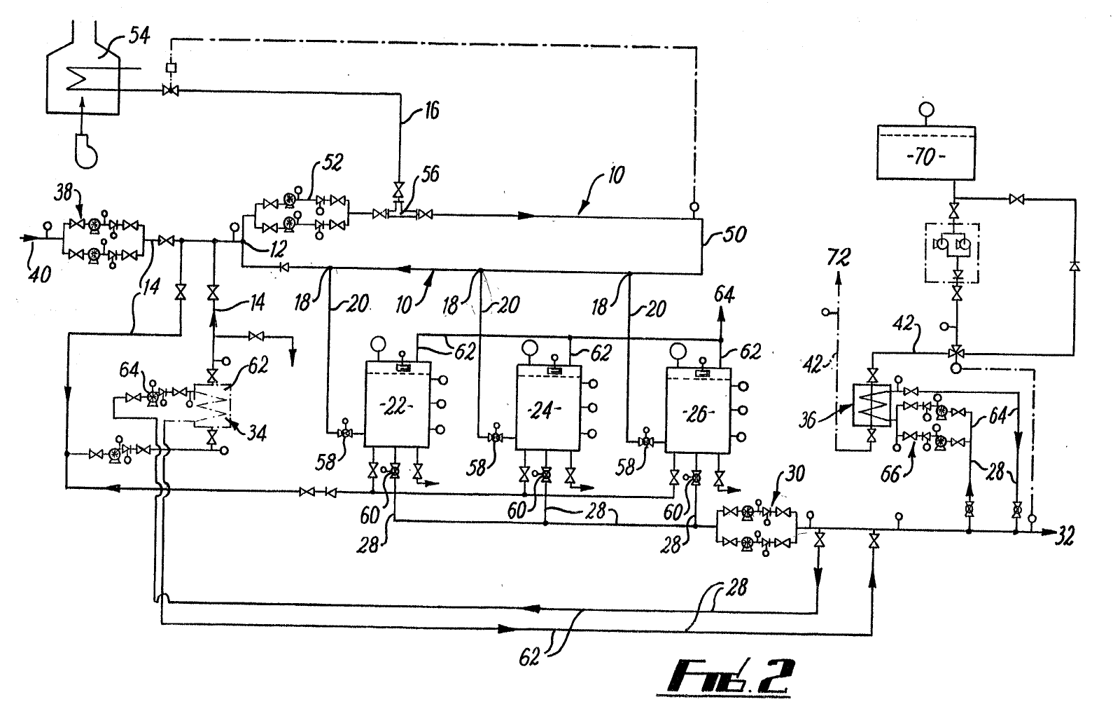

FIG. 2 shows a more specific circuit diagram of a sludge heat treatment apparatus; and

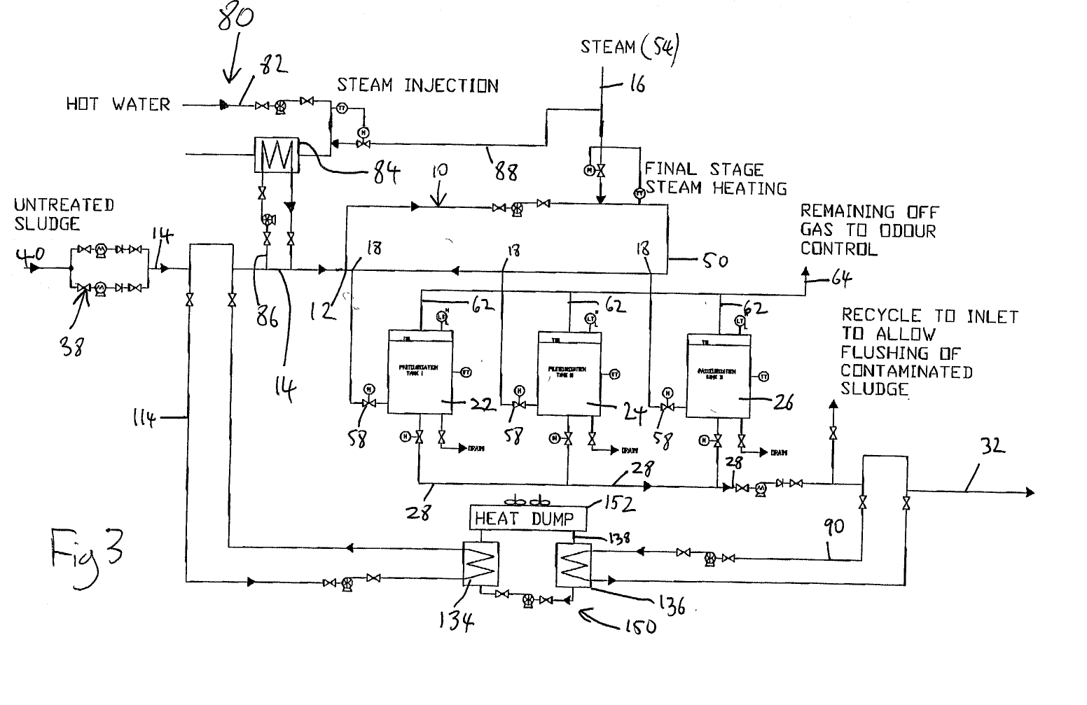

FIG. 3 shows a circuit diagram of an embodiment of a heat treatment apparatus in accordance with the present invention.

FIG. 1 shows for reference a known treatment vessel or thermal reactor 10 to which raw sewage sludge to be treated is fed through an inlet 12 by way of an inlet line 14. A heating medium which in the embodiment is steam is introduced into the reactor by way of a line 16 to heat the sludge in the reactor.

The input of heating medium and the form of the thermal reactor will be described in greater detail below but it should be realised that untreated sludge is fed continuously through the inlet 14 and heated sludge is removed continuously at effectively the same rate from the outlet 18 and through a distribution pipe 20 to one of three, essentially identical insulated heat treatment tanks 22, 24, 26. The block diagram of FIG. 1 does not show arrangement of pumps, valves and control assemblies which control the flow of sludge through the pipe 20 in such a way that heated sludge is supplied to a first tank, for example 22 while a second tank, for example 24 is discharging sludge at effectively the same rate. The remaining tank 26, during this filling/emptying process is holding heated sludge therein so that heat treatment can be completed, the sludge being discharged from tank 24 having already experienced a complete heat treatment.

A duct 28 for discharging heat treated sludge has a pump assembly 30 incorporated therein and before treated sludge is discharged to digesters shown diagrammatically in the block diagram of FIG. 1 by the reference numerals 32 heat is recovered therefrom by passing the sludge through two heat exchangers 34, 36. In the first heat exchanger 34 hot treated processed sludge in the duct 28 is brought into heat exchange relationship with cold incoming untreated sludge in the duct 14, raw sludge being supplied from a holding arrangement 40 by a pump 38 interposed in the inlet line 14.

In the second heat exchanger 36 further heat is removed from the heat treated sludge by passing it in heat exchange relationship with a cooling fluid, which is conveniently water, fed and discharged through a line 42.

The present invention realises that there are disadvantages in heating sludge by passing steam therethrough unless the steam and sludge are mixed in predetermined, controlled conditions. These conditions are achieved by the apparatus shown in the circuit diagram illustrated in FIG. 2 where the same reference numerals have been used for components similar to those described with reference to FIG. 1.

The thermal reactor 10 comprises a pipe 50 of approximately 100 mm diameter arranged in a closed loop and having an assembly of pumps 52 interposed therein, each pump having suitable valve and control assemblies which did not form part of the present invention and will not be described but the pumps are such that sludge is passed around a continuous loop at a relatively high velocity, for example around 20 liters per second. Sludge is introduced into the loop at inlet 12 by way of a supply line 14 having a pump assembly 38 interposed therein.

Heating steam is supplied to the pipework loop 50 from a steam boiler 54 through a supply line 16 and a steam injector 56 which provides steam bubbles in the flow of sludge, the bubbles being around 200 microns in diameter. This steam injected, coupled with the high sludge flow rate through the pipe 50 gives rise to highly turbulent conditions whereby transfer of heat from the condensing steam bubble to the sludge is efficiently and evenly achieved without mechanical or sonic disruption as the bubbles collapse. Heat transfers is good as a result of the high surface and of the plurality of small diameter bubbles. Ideally, the volume of steam added should be limited for thermal efficiency and to reduce volume increase due to steam condensation.

The heated sludge must be maintained at or around this temperature of around 70° C. for a predetermined period of time to allow the pasteurisation process to be concluded. Three insulated non-pressurised holding tanks 24,26,28 are provided each connected at outlets 18 by pipes 20 to the pipework loop 50 the pipes 20 supplying heated sludge to or near to the base of each tank. Valves 58 and appropriate control means are provided in each supply line 20 and are activated such that at any one time one valve is opened so that only one tank is being filled at any particular time. As this tank is being filled one of the remaining two tanks is being emptied at the same rate through a discharge valve 60 into an outlet pipe 28 through which heat treated sludge is pumped by pump assembly 30. During a filling/emptying cycle the remaining tank is neither filled nor emptied so that heated sludge is held therein while the pasteurisation process is completed.

In practice the pipe loop 50 is located alongside the tanks near to their bases and are of the loop serving as an outlet manifold.

The air space at the top of each tank is connected to that in the other two tanks by means air lines 62 such that on filling the removed air is transferred to the emptying tank thereby containing odours etc. within a closed system. The line 60 has a discharge vent 64 fitted with an odour control (not shown).

Means are provided for removing heat from the treated sludge passing along outlet duct 28 to digestors 32. The means comprise a first heat exchanger 34 through which passes a sludge loop 62 which taps off and returns sludge from and to the outlet line 28, the loop 62 having a pump 64 therein to encourage flow of sludge. The coolant for the heat exchanger 34 is untreated sludge tapped off and returned to the sludge supply line 14 between the pump assembly 38 and the sludge inlet 12.

The second heat exchanger 36 similarly taps off sludge from the outlet pipe 28 through a sludge loop 64 having a pump assembly 66 therein, the coolant being a suitable liquid which is passed through a line 42 from a supply 7 and is passed to waste 72.

It will be realised that the process and apparatus described above, particularly with reference to the circuit illustrated by FIG. 2 provides for the efficient pasteurisation of sewage sludge. The high flow rate of sludge through the pipework loop 50 caused by the pumping assembly 52 coupled with the injection of a plurality of small diameter bubbles by the injector 56 creates a Reynolds number flow condition which results in very high turbulence and consequently efficient transfer of heat from steam to the sludge. As the bubbles are small they condense rapidly but, by the same token, because they are small a bubble collapse gives minimal effects, for example, the noise problems created in other low flow/high bubble sized systems are mitigated. The arrangement is compact and can be located alongside the three storage tanks thereby reducing the space required and as the return run of the pipework loop contains all three outlets 18 it serves as an outlet manifold thereby decreasing the pipework involved. Efficient recycling is achieved by use of, especially, the first heat exchanger 34 where heat from the heat treated sludge is transferred to untreated sludge. The final cooling of the heat treated sludge can be achieved using any suitable cooling medium, for example treated effluent. As the system is effectively a closed system, dispersal of undesirable odours into the ambient air is reduced to a minimum, the linking of the air in the storage tanks above the sludge level with the other neighbouring tanks enhancing this advantage. It will be further realised that at least the storage operation is carried out at atmospheric pressure so that there is no need to provide pressurised storage tanks thereby introducing the difficulties and relatively high expense following from the use of pressure vessels.

Referring to FIG. 3, there is shown apparatus in accordance with the present invention. The embodiment shown in FIG. 3 comprises many of the same features as those shown in FIG. 2, and these have been designated with the same reference numeral. The apparatus depicted in FIG. 3 is directed to regulating the volume of steam injected to the sludge for treatment and subsequently controlling the output temperature of treated processed sludge.

The difference between the embodiment shown in FIG. 3 and that shown in FIG. 2 is that, with the embodiment shown in FIG. 3, the untreated sludge is heated by a heating assembly 80 in which hot water is fed via lines 82 to a heat exchanger 84 to heat sludge past via lines 86 from the inlet line 14. The hot water through the lines 82 may be from suitable heating means, for example a boiler, or combined heat and power plant and will typically constitute unused or spare heating capacity. In addition to hot water fed along the lines 82, steam is extracted from the steam heating line 16 via a line 88 and is injected into the line 82 for further heating. The pre-heated sludge is then passed from the heat exchanger via the line 86 to the line 14 to be fed to the pipework loop 50 by the inlet 12.

The heat exchanger 84 heats the sludge from a temperature of about 30° C. to a temperature of about 55° C. However, where heating capacity is available, the temperature may be elevated to a higher temperature in order to limit steam requirements later or so that the treatment temperature is more quickly achieved.

Prior to feeding the sludge via the line 86 to the heat exchanger 84, the sludge is passed via a line 114 to a heat exchanger 134 for initial pre-heating from a temperature of about 10C to a temperature of about 30° C. The sludge is passed from the heat exchanger 134 to the line 14 where it can be extracted via the lines 86 as described above.

It will be appreciated that the heat exchanger 134 is essentially involved with heat recovery from the still hot processed sludge. The degree of heating will depend upon the output processed sludge temperature, incoming untreated sludge temperature and the sludge flow rate. The heat exchanger 84 as outlined elevates the incoming untreated sludge to a temperature of approximately 55° C. Thus, the steam injected into the incoming untreated sludge need only elevate that sludge to a temperature above 70° C. in order to initiate the treatment process in the reactors 22,24,26. In such circumstances, a lower volume of injected steam is required in order to achieve this necessary treatment temperature. The steam injected is essentially regulated dependent upon the incoming untreated sludge temperature to achieve the desired treatment temperature which is normally above 70° C. The present apparatus utilises pre-heating derived from recovery of heat out of the still hot processed sludge and available typically unused or spare heating capacity to further heat the incoming untreated sludge prior to regulated injection of steam for final elevation to the treatment temperature. Clearly, the amount of recovered heat from the processed treated sludge and available unused or spare capacity may vary such that the volume of regulated injected steam may also vary in order to achieve the treatment temperature. Nevertheless, typically the volume of injected steam will be limited for thermal efficiency and also to reduce the sludge volume increase due to steam condensate adding to the treated sludge. Generally, surplus heat, whether it be from unused or from spare heat source capacity, will be in a form more readily convertible to hot liquid such as water. This hot water is coupled through feedlines and appropriate valving to the heat exchanger 84 in order to provide the elevation in incoming untreated sludge temperature to limit or adjust that incoming untreated sludge temperature for consistency with the regulated volume of steam injected into the flow of sludge for treatment in the reactors.

It will be understood, where there is surplus steam capacity that surplus capacity may be utilised in two ways. Firstly, a greater volume of steam may be injected directly into the flow of sludge in order to achieve the treatment temperature, but possibly increase the treated sludge volume due to steam condensate and/or present pressure regulation and safety problems. Alternatively, the spare steam capacity may be converted through a suitable heat exchanger condenser such that the still hot condensed steam as hot water can be used through an appropriate feed to the heat exchanger 84. Thus, the incoming untreated sludge has its temperature elevated as desired and the “ideal” volume of injected steam used for heat elevation to the treatment temperature whilst limiting increased sludge volume due to steam condensate, etc. By this alternative use of surplus steam it will be appreciated that the incoming untreated sludge is more progressively heated such that there is less caking of the sludge upon the heat exchanger surface interface due to a high temperature gradient thereacross from the untreated sludge to the hot water transfer medium.

The heat exchanger 134 is part of a further heat exchange arrangement 150 which will be described in more detail below. Thus, the incoming sludge is initially heated by two heat exchangers 84, 134, the heat exchanger 134 being provided to recover heat from the treated sludge, and the heat exchanger 84 being provided to use heat from elsewhere on the plant, for example a hot water boiler, an engine, or a combined heat and power plant. In addition, the heat exchanger 84 can utilise steam from the boiler 54.

A further modification is that the apparatus shown in FIG. 3 does not use the two heat exchangers 34, 36 as described with reference to FIG. 2. Instead, a combined heat exchange arrangement or assembly 150 is provided.

The heat exchange arrangement 150 is in the form of a sludge-water-sludge heat exchanger, in which treated sludge passing via the lines 28 from the holding tanks 22, 24, 26 is extracted from the discharge line 32 via a line 90 to be passed to the heat exchange assembly 150.

The heat exchange assembly 150 which comprises a heat dump device 152, the heat exchanger 134, and a further heat exchanger of 136.

Water from the heat dump device 152 passes via a line 138 to one side of the heat exchanger 136. Sludge passing through the other side of the heat exchanger 136 via the line 90 passes heat to the water. The heated water passes from the heat exchanger 138 to the heat exchanger 134 passing on one side, and the sludge which passes on the other side from the line 114 extracts heat from the water. The water then passes to the heat duct device 152 where heat is transferred across a plate exchanger to water or air on the other side thereof. Thus, the heat exchange assembly 150 enables heat from the treated sludge passing through the line 90 to be transferred to untreated sludge passing through the line 114. The use of the heat exchange assembly 150 to replace the heat exchangers 34, 36 of the embodiment shown in FIG. 2 has the advantage that a single heat exchange unit is required to recover heat to the untreated sludge and to allow excess heat to be dumped. Moreover, heat dumped at the heat dump device occurs where the temperatures are relatively high, thereby minimising the size of the heat dump device.

The temperature of the heated sludge discharged via the discharge line 32 can be varied by adjusting the amount of heat dumped at the heat dump device.

A single heat exchanger assembly 150 as described above achieves the objectives of heat recovery from the treated sludge along with control of output treated sludge temperature. Essentially, the heat exchanger assembly 150 comprises two heat exchangers acting through, or being immersed in, a common heat dump such as a volume of water. Thus, the heat exchanger 136 presents the hot treated sludge to the heat dump 152 in order that at least a proportion of its heat energy is liberated to that heat dump 152. The heat dump 152 then presents that heat energy through an elevation in its own temperature to the heat exchanger 154 which in turn provides initial pre-heating to the incoming untreated sludge as described previously. In accordance with this further refinement of the present invention the heat dump 152 itself can disperse through an appropriate heat sink or radiator structures (not shown) a proportion of the heat energy taken from the hot treated sludge through heat exchanger 136. In such circumstances, the final temperature of the treated sludge presented through discharged line 32 can be controlled to a desired value and through the heat dump 152 the amount of heat energy taken by the heat exchanger 134 in order to initially pre-heat the incoming untreated sludge from line 14 can also be controlled. The heat exchanger 150 provides two separate functions, heat recovery from the processed sludge and final processed sludge temperature control by changing the water temperature in the heat dump 152 thereby altering the amount of heat liberated through a temperature gradient variation. It will also be appreciated that the rate of heat dump transfer medium flow will affect the heat recovery and final temperature functions of the assembly 150 and this will be dependant upon pumping rate, etc.

Various modifications can be made without departing from the scope of the invention, for example the pipework assembly of pumps control arrangement can be modified in any suitable manner. Additionally more than three storage tanks can be employed and heat exchange arrangements can be modified.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1. A method of treating sludge by continuously passing a flow of sludge through a thermal reactor with a regulated injection of steam into the flow of sludge in order to achieve a treatment temperature, the rate of sludge flow and manner of steam injection being such that heat treatment conditions are provided, removing heated sludge from the reactor and replacing the removed sludge by untreated sludge, incoming untreated sludge being pre-heated as required from any available heat source through heat exchange with an appropriate transfer medium in order to regulate, and normally limit, the volume of steam injection required to achieve the treatment temperature in the flow of sludge or the speed of attainment of the treatment temperature.

2. A method as claimed in claim 1, in which the sludge is caused to flow in a continuous pipework loop from a sludge inlet to a sludge outlet spaced from the inlet, steam being injected into the sludge downstream of the sludge inlet and closer to the inlet than the outlet.

3. A method as claimed in claim 1, in which the volume of steam is of the order of one hundred times that of the sludge into which it is injected.

4. A method as claimed in claim 1, in which treated sludge is collected sequentially in at least three collection tanks.

5. A method as claimed in claim 4, in which while one tank is filling another is emptying and a third is storing heated sludge during its heat treatment cycle.

6. A method as claimed in claim 4, in which the tanks are maintained at atmospheric pressure.

7. A method as claimed in claim 4, in which sludge is fed to the tanks at or near the bottom of the tanks.

8. A method as claimed in claim 5, in which the air discharging from a filling tank is fed to an emptying tank.

9. A method as claimed in claim 4, in which treated sludge discharged from a tank is cooled in a two stage process in separate heat exchangers.

10. A method as claimed in claim 9, in which heat is removed from treated sludge in a first heat exchanger and transfers to unprocessed untreated sludge to be fed to the reactor.

11. A method as claimed in claim 9, in which heat is removed from treated sludge in the second stage heat exchanger by a cooling fluid.

12. A method as claimed in claim 1 wherein sludge removed from the thermal reactor is subject to a heat transfer step to recover heat to the incoming untreated sludge prior to the pre-heating step.

13. A method as claimed in claim 12 wherein the recovery step comprises arranging the heated sludge in a heat transfer relation with a heat absorption medium.

14. A method as claimed in claim 13 wherein the heat recovery step includes arranging the incoming untreated sludge in heat transfer relation with said heat absorption medium such that heat is transferred from the treated sludge to the heat absorption medium and thereafter to the incoming untreated sludge.

15. A method as claimed in claim 12 wherein after the heat recovery step, the heat absorption medium transfers heat to a heat dump medium.

16. Apparatus for treating sludge comprising a thermal reactor, means for circulating sludge continuously around the reactor as a flow of sludge, means for regulating injection steam into the flow of sludge, means for removing heated sludge from the reactor and means for replacing removed heated sludge by untreated sludge, the apparatus also includes a heat exchange assembly whereby heat is transferred between the heated sludge and the incoming untreated sludge with pre-heating means for heating the incoming untreated sludge before that sludge is heated in the thermal reactor to regulate the volume of steam required to be injected into the flow of sludge in order to heat that sludge in the reactor to a treatment temperature or the speed of attainment of that treatment temperature by the sludge.

17. Apparatus as claimed in claim 16 in which the thermal reactor is a continuous pipework loop having an inlet and at least one outlet spaced from the inlet, a steam injector for providing heating steam to the sludge flow in the form of small bubbles being provided adjacent the inlet.

18. Apparatus as claimed in claims 16, in which three collection tanks are provided for collecting heated sludge, each tank having inlet and outlet means and control means for said inlet and outlet means whereby as one tank is being filled with heated sludge, another is being emptied of heat treated sludge, the or each remaining tank storing the sludge for complete heat treatment during said filling/emptying process.

19. Apparatus as claimed in claim 18, in which said tanks are maintained at atmospheric pressure by providing a connection between atmosphere and the air space at the top of each tank.

20. Apparatus as claimed in claim 18, in which the sludge inlets and outlets are located at or near the bottom of each tank.

21. Apparatus as claimed in claim 16, in which heat exchange means are provided in the outlet line from said tanks.

22. Apparatus as claimed in claim 21, in which a further two heat exchangers are provided, the first heat exchanger exchanging heat between heat treated sludge and untreated sludge and the second, downstream, heat exchanger exchanging heat from heat treated sludge an a cooling fluid.

23. Apparatus as claimed in claim 16, in which the pipework loop is arranged alongside the tanks at or near the base thereof.

24. An apparatus as claimed in claim 16 wherein the heat exchange assembly in addition to transferring heat between the treated sludge and the incoming untreated sludge also exchanges heat with a cooling medium.

25. An apparatus as claimed in claim 16 wherein the heat exchange assembly comprises a first heat exchanger in which sludge is arranged in thermal communication with a heat transfer medium and means are provided to feed treated sludge to the first heat exchanger in order to transfer heat from the treated sludge to the heat transfer medium whilst a second heat exchanger also in thermal communication with the heat transfer medium is associated with means to provide a feed of incoming untreated sludge to the second heat exchanger to transfer heat from the heat transfer medium to the incoming untreated sludge.

26. Apparatus as claimed in claim 16 wherein the heat exchange assembly includes circulating means to circulate the heat transfer medium between the first and second heat exchangers.

27. Apparatus as claimed in claim 16 wherein the heat exchange assembly includes heat dump means in association with the heat transfer medium such that heat can be transferred from the heat transfer medium to the heat dump medium.

28. Apparatus as claimed in claim 27 wherein the heat dump means comprises a heat exchange plate across which heat transfer from the heat transfer medium to the heat dump medium is facilitated.

29. Apparatus as claimed in claim 27 wherein circulating means is provided to circulate heat transfer medium through the first heat exchanger, second heat exchanger and the heat dump means.

30. Apparatus as claimed in claim 16 wherein the pre-heating means for the incoming untreated sludge is provided prior to the reactor.

31. Apparatus as claimed in claim 16 wherein the pre-heating means includes means for heating a heating medium and heat exchange means for transferring heat from the heating medium to the incoming untreated sludge.

32. Apparatus as claimed in claim 31 wherein the heating medium is heated by steam from steam means.

33. Apparatus as claimed in claim 32 wherein the steam means is in fluid communication with the means for injecting steam into the flow of sludge.

34. Apparatus as claimed in claim 16 wherein the pre-heating means includes further heat transfer means to transfer heat from the treated sludge to the incoming untreated sludge.

35. Apparatus as claimed in claim 34 wherein the further heat transfer means includes the second heat exchanger.

36. Any novel subject matter or combination including novel subject matter disclosed herein, whether or not within the scope of or relating to the same invention as any of the preceding claims.
